(19) United States
(12) Patent Application Publication
(10) Pub. No.: US 2019/0133849 A1
(43) Pub. Date: May 9, 2019

(54) DISPOSABLE GLOVE FOR THE HYGENIENIC REMOVAL AND DISPOSAL OF FEMININE PROTECTION PRODUCTS

(71) Applicant: Suzanne Marie Geer, Saylorsburg, PA (US)

(72) Inventor: Suzanne Marie Geer, Saylorsburg, PA (US)

(21) Appl. No.: 16/229,129

(22) Filed: Dec. 21, 2018

Related U.S. Application Data
(60) Provisional application No. 62/579,858, filed on Oct. 31, 2017.

(51) Int. Cl. A61F 13/551; A41D 19/00

(52) U.S. Cl. CPC A61F 13/5515; A41D 19/0075; A61F 13/5518; A61F 2013/55155; A41D 2400/52; A41D 19/0062; A41D 19/002

(57) ABSTRACT

A sealable, disposable hand cover to facilitate the sanitary handling and disposal of feminine protection products, and thereby eliminating odor and spreading of germs onto surrounding surfaces during such disposal is provided. The sealable, disposable hand cover provides a sealing mechanism at or just inward of the opening, where a user slides their hand, so that when the sealable, disposable hand cover is reverse the feminine protection product that was handled by the sealable, disposable glove can be captured therein and sealed closed along or near the opening. Then the sealable, disposable hand cover may be odorlessly disposed of, feminine protection product and all.

### CROSS-REFERENCE TO RELATED APPLICATION

This application claims the benefit of priority of U.S. provisional application No. 62/576,858, filed 25 Oct. 2017, the contents of which are herein incorporated by reference.

### BACKGROUND OF THE INVENTION

The present invention relates to feminine hygiene products and, more particularly, to a disposable hand cover for the hygienic removal and disposal of feminine protection products.

Every day women are faced with the problem of disposing of feminine hygiene products that involve bodily fluids in a hygiene manner. Public restrooms are filled with germs, and so it is widely-assumed that any product provided by such restrooms for disposal of feminine hygiene products are themselves unsanitary. Unfortunately, there currently is no one easily totable article adapted to hygienically handle, seal away and dispose of used feminine hygiene products.

As can be seen, there is a need for a convenient and hygienic product for the easy handling and disposal of feminine hygiene products, that users can discretely tote on their person. By not handling used feminine products with bare hands, the present invention eliminates the spread of bacteria. Advantageously, because the present invention seals in odor, pets are not tempted to rummage through garbage, and drag embarrassing items throughout a house.

### SUMMARY OF THE INVENTION

In one aspect of the present invention, a sealable, disposable hand cover for both handling a disposing of an item wherein the item is encapsulated and sealing in the inverted hand cover after being handled thereby, the sealable, disposable hand cover includes the following: a hand cover providing an interior surface defining a first cavity dimensioned and adapted to receive a human hand through an opening in the hand cover; the interior surface having an opposing exterior surface so that when hand cover is inverted a second cavity is formed, the second cavity being generally coextensive as the first cavity; and a sealing mechanism provided by the hand cover at or just inward of said opening, the sealing mechanism movable between an unsealed condition and a sealed condition closing off said opening.

These and other features, aspects and advantages of the present invention will become better understood with reference to the following drawings, description and claims.

### BRIEF DESCRIPTION OF THE DRAWINGS

FIG. 1 is an elevational view of an exemplary embodiment of the present invention;

FIG. 2 is perspective view of an exemplary embodiment of the present invention, shown in use handling a used sanitary pad;

FIG. 3 is perspective view of an exemplary embodiment of the present invention, shown in use being folded over the user's hand while grasping the used sanitary pad;

FIG. 4 is perspective view of an exemplary embodiment of the present invention, shown folded over the used sanitary pad, and pulled of the user's hand;

FIG. 5 is an elevational view of an exemplary embodiment of the present invention shown containing the sanitary pad as the present invention is being sealed at the opening;

FIG. 6 is perspective view of an exemplary embodiment of the present invention, shown being disposed of;

FIG. 7 is an elevational view of an exemplary embodiment of the present invention shown with the present invention providing finger compartments;

FIG. 8 is a detailed elevational view of an exemplary embodiment of the present invention shown with an adhesive strip sealing mechanism; and

FIG. 9 is a detailed elevational view of an exemplary embodiment of the present invention shown with a cinch string sealing mechanism.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a sealable, disposable glove to facilitate the sanitary handling and disposal of feminine protection products, and thereby eliminating odor and spreading of germs onto surrounding surfaces during such disposal. The sealable, disposable glove provides a sealing mechanism at or just inward of the opening, where a user slides their hand, so that when the sealable, disposable glove is reverse the feminine protection product that was handled by the sealable, disposable glove can be captured therein and sealed closed along or near the opening. Then the sealable, disposable glove may be odorlessly disposed of, feminine protection product and all.

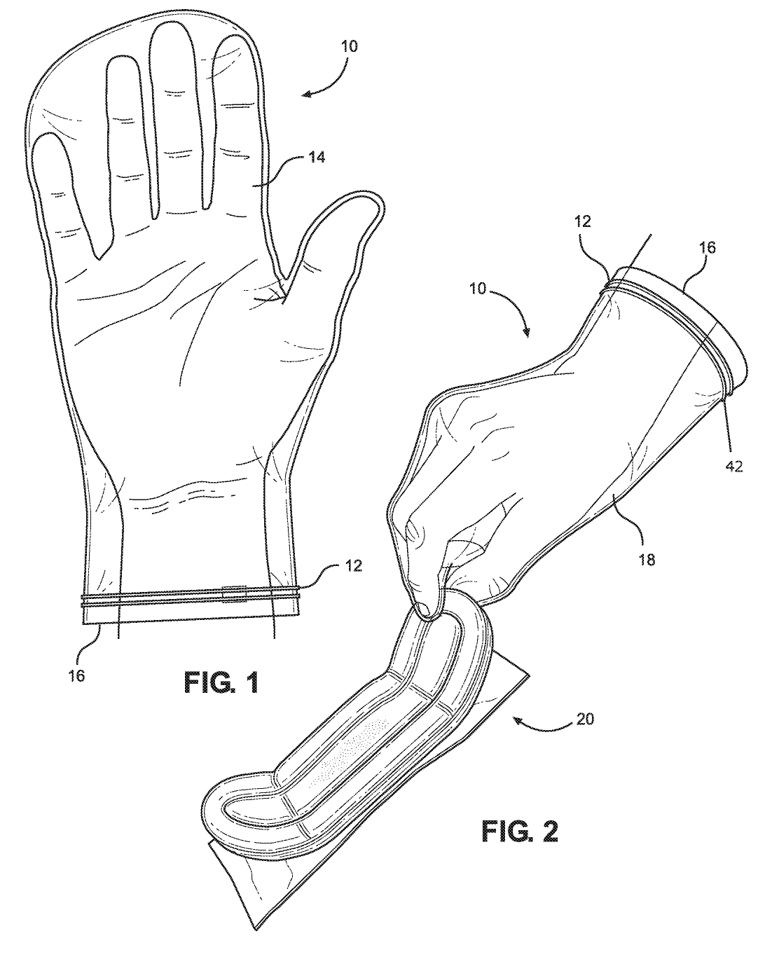

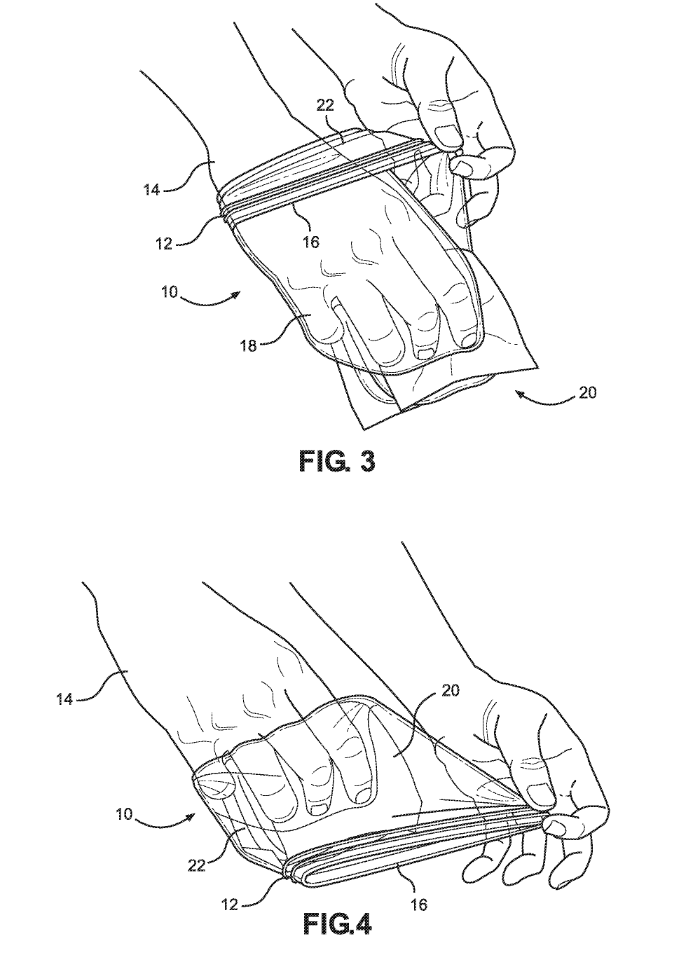

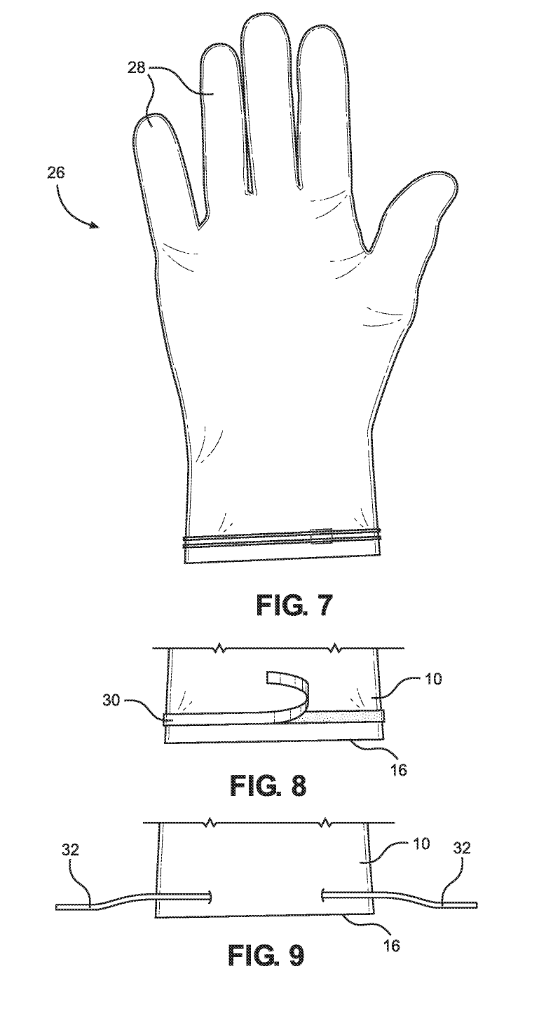

Referring to FIGS. 1 through 9, the present invention may include a sealable, disposable hand cover 10 to facilitate the sanitary handling and disposal of feminine protection products 20, such as a used pad or a tampon, into a proper waste receptacle 24. The glove 10 provides an exterior surface 18 and interior surface 22, the interior surface 22 defining a cavity dimensioned an adapted to receive a human hand 14. In certain embodiment, the sealable, disposable hand cover 10 may provide separate finger components 28 for all or most human digit, “glove” style 26. While in other embodiments the cavity will conform to “mitten” style, without separate finger compartments 28.

In either embodiment, the cavity communicates with an opening 14 for receiving said human hand 14. Along the exterior surface 18 and/or the interior surface 22 adjacent to the opening 16, possibly dispose just inward of a periphery of said opening 16 may be a sealing mechanism 40 for sealing closed the opening 16 or otherwise preventing a fluid connection between the cavity and the opening 16. The sealing mechanism 40 may include a zipper seal 12 having spaced apart tracks 42 disposed along the exterior surface 18 and/or the interior surface 22. In general, the sealing mechanism 40 is movable between an unsealed and a sealed condition.

Referring to FIGS. 8 and 9, the sealing mechanism 40 may include one or more adhesive strips 30 or cinch strings 32 disposed along the exterior surface 18 and/or the interior surface 22.

The sealable, disposable hand cover 10 may be made from various plasticized, rubber, and/or hypoallergenic, non-toxic material that prevents passage of microbes, pathogens and/or odors.

The sealable, disposable hand cover 10 is reversible as illustrated in the process embodied in FIGS. 2 through 6. The sealing mechanism 40 may be disposed along the exterior surface 18 and/or the interior surface 22 in light of the reversibility and the process of use illustrated in FIG. 2 through 6.

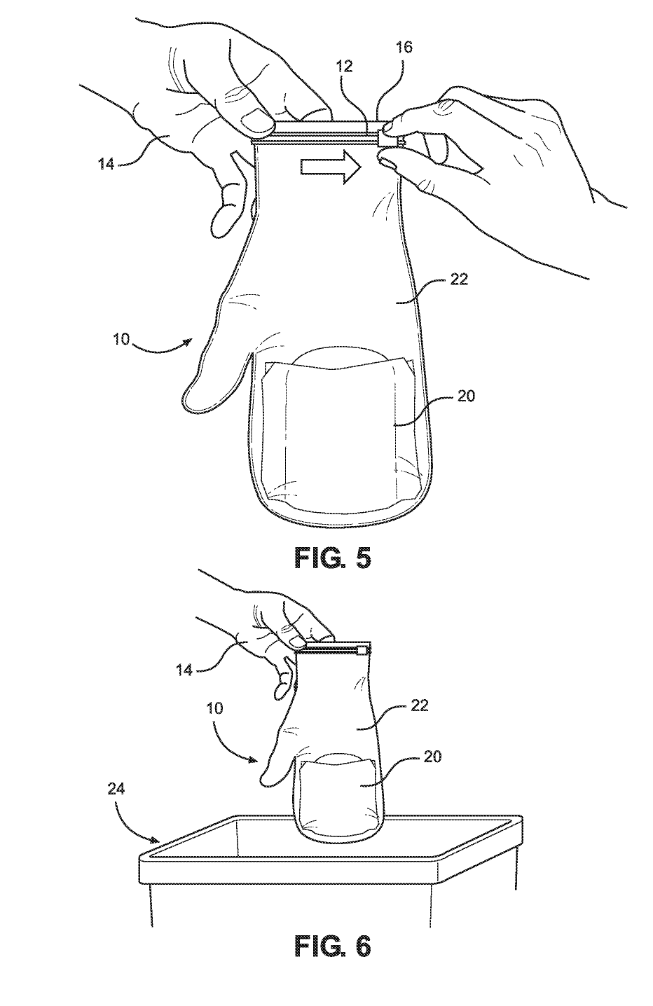

A method of using the present invention may include the following. The sealable, disposable hand cover 10 disclosed above may be provided. A user may don the sealable, disposable hand cover 10 over their hand 14 to remove and dispose of a feminine hygiene product 20. Either privately at home or at a public restroom without touching it with their bare hands. The user then inverts the sealable, disposable hand cover 10, and seals it with the sealable closure. As shown in FIG. 6, the sealed sealable, disposable hand cover 10 has captured the feminine hygiene product 20 in a new cavity formed by the inversion/reversal of the sealable, disposable hand cover 10. Then the user engages the sealing mechanism 40 into the sealed condition, and simply disposes of the sealable, disposable hand cover 10 and encapsulated feminine hygiene product 20 in a proper receptacle 24. In certain embodiments, the sealable, disposable hand cover 10 may be transparent so that a user can be assured they have imprisoned their target in the newly formed inverted cavity. The sealable, disposable hand cover 10 may also may be various colors for aesthetic, branding, identification purposes or the like.

As a result, no odor is allowed to escape the sealed inverted cavity and the hand 14 are advantageously left clean. Because there is no odor, pets are not attracted to the products and thus, do not drag the product out of the trash. In one embodiment, the sealable, disposable hand cover 10 may be scented.

Alternatively, the sealable, disposable hand cover 10 can also be used to dispose of diapers or adult incontinence products, or to clean up food in the kitchen, for example, to avoid the spread of salmonella.

Advantageously, users can conveniently store the sealable, disposable hand cover 10, which are dimensioned to fit various-sized hands, in their purse or pocket. The sealable, disposable hand cover 10 may also be installed in public restroom dispensers and made available wherever feminine products are sold. The sealable, disposable hand cover 10 may be located by baby changing tables or accessible in each bathroom stall.

It should be understood, of course, that the foregoing relates to exemplary embodiments of the invention and that modifications may be made without departing from the spirit and scope of the invention as set forth in the following claims.

What is claimed is:

1. A sealable, disposable hand cover for both handling a disposing of an item wherein the item is encapsulated and sealing in the inverted hand cover after being handled thereby, comprising:
a hand cover providing an interior surface defining a first cavity dimensioned and adapted to receive a human hand through an opening in the hand cover;
the interior surface having an opposing exterior surface so that when hand cover is inverted a second cavity is formed, the second cavity being generally coextensive as the first cavity; and
a sealing mechanism provided by the hand cover at or just inward of said opening, the sealing mechanism movable between an unsealed condition and a sealed condition closing off said opening.

2. The sealable, disposable hand cover of claim 1, wherein the hand cover provides finger compartment for most or all of said human hand.

3. The sealable, disposable hand cover of claim 1, wherein the hand cover is translucent or transparent.

4. The sealable, disposable hand cover of claim 1, wherein the sealing mechanism is a zipper seal disposed along the exterior surface.

5. The sealable, disposable hand cover of claim 1, wherein the sealing mechanism is a zipper seal disposed along the interior surface.

6. The sealable, disposable hand cover of claim 1, claim 1, wherein the sealing mechanism is one or more adhesive strip disposed along the exterior surface.

7. The sealable, disposable hand cover of claim 1, wherein the sealing mechanism is one or more adhesive strip disposed along the interior surface.

8. The sealable, disposable hand cover of claim 1, claim 1, wherein the sealing mechanism includes cinch string disposed along the exterior surface.

9. The sealable, disposable hand cover of claim 1, claim 1, wherein the sealing mechanism includes cinch string disposed along the interior surface.
